(19) United States
(12) Patent Application Publication
(10) Pub. No.: US 2018/0085597 A1
(43) Pub. Date: Mar. 29, 2018

(54) BRACHYTHERAPY APPLICATOR WITH RADIATION SENSORS

(71) Applicant: RADIADYNE LLC, Houston, TX (US)

(72) Inventors: John ISHAM, Houston, TX (US); Eric HYMAN, Houston, TX (US)

(21) Appl. No.: 15/713,123

(22) Filed: Sep. 22, 2017

Related U.S. Application Data
(60) Provisional application No. 62/399,407, filed on Sep. 25, 2016.

(51) Int. Cl. A61N 5/10

(52) U.S. Cl. CPC A61N 5/1014; A61N 5/1048; A61N 2005/1092; A61N 5/1007

(57) ABSTRACT

A uni- or multi-channel cylinder brachytherapy applicator is combined with real-time radiation sensor cables, as well as methods of making and using same.

### PRIOR RELATED APPLICATIONS

This application claims priority to U.S. Ser. No. 62/399,407, filed Sep. 25, 2016, and incorporated by reference herein in its entirety for all purposes.

### FIELD OF THE INVENTION

The invention relates to brachytherapy applicators and radiation sensors that are used for brachytherapy.

### BACKGROUND OF THE INVENTION

“Brachytherapy (from the Greek word brachys, meaning “short-distance”), al so known as internal radiotherapy, sealed source radiotherapy, curietherapy or endocurietherapy, is a form of radiotherapy where a sealed radiation source is placed inside or next to the area requiring treatment. Brachytherapy is commonly used as an effective treatment for cervical, prostate, breast, and skin cancer and can also be used to treat tumors in many other body sites.

Different types of brachytherapy can be defined according to (1) the placement of the radiation sources in the target treatment area, (2) the rate or ‘intensity’ of the irradiation dose delivered to the tumor, and (3) the duration of dose delivery.

The two main types of brachytherapy treatment in terms of the placement of the radioactive source are interstitial and contact. In the case of interstitial brachytherapy, the sources are placed directly in the target tissue of the affected site, such as the prostate or breast. Contact brachytherapy involves placement of the radiation source in a space next to the target tissue. This space may be a body cavity (intracavitary brachytherapy) such as the cervix, uterus or vagina; a body lumen (intraluminal brachytherapy) such as the trachea or oesophagus; or externally (surface brachytherapy) such as the skin. A radiation source can also be placed in blood vessels (intravascular brachytherapy) for the treatment of coronary in-stent restenosis.

The dose rate of brachytherapy refers to the level or ‘intensity’ with which the radiation is delivered to the surrounding medium and is expressed in Grays per hour (Gy/h). Low-dose rate (LDR) brachytherapy involves implanting radiation sources that emit radiation at a rate of up to 2 Gy·h−1. LDR brachytherapy is commonly used for cancers of the oral cavity, oropharynx, sarcomas and prostate cancer. Medium-dose rate (MDR) brachytherapy is characterized by a medium rate of dose delivery, ranging between 2 Gy·h−1 to 12 Gy·h−1. High-dose rate (HDR) brachytherapy is when the rate of dose delivery exceeds 12 Gy·h−1. The most common applications of HDR brachytherapy are in tumors of the cervix, esophagus, lungs, breasts and prostate.

Pulsed-dose rate (PDR) brachytherapy involves short pulses of radiation, typically once an hour, to simulate the overall rate and effectiveness of LDR treatment. Typical tumor sites treated by PDR brachytherapy are gynecological and head and neck cancers.

The placement of radiation sources in the target area can be temporary or permanent. Temporary brachytherapy involves placement of radiation sources for a set duration (usually a number of minutes or hours) before being withdrawn. The specific treatment duration will depend on many different factors, including the required rate of dose delivery and the type, size and location of the cancer. In LDR and PDR brachytherapy, the source typically stays in place up to 24 hours before being removed, while in HDR brachytherapy this time is typically a few minutes.

Permanent brachytherapy, also known as seed implantation, involves placing small LDR radioactive seeds or pellets (about the size of a grain of rice) in the tumor or treatment site and leaving them there permanently to gradually decay. Over a period of weeks or months, the level of radiation emitted by the sources will decline to almost zero. The inactive seeds then remain in the treatment site with no lasting effect. Permanent brachytherapy is most commonly used in the treatment of prostate cancer.

Intracavity brachytherapy is used in body cavities, such as the vagina, rectum, and the like. Although there are many types of brachytherapy applicators, one common applicator is a solid cylindrical tube with one or more slender hollow channels therein for placement of radiation. Using vaginal brachytherapy applicators as an example, we will describe the applicators in use.

The most commonly used applicator for intracavitary vaginal brachytherapy is single channel vaginal cylinder. However, due to its radial symmetry of dose distribution, a single channel applicator offers limited possibilities to optimize the treatment plan according to the patient's anatomy. Aiming to improve the capabilities of vaginal brachytherapy, multichannel applicators have been developed. The additional channels at the periphery of the applicator support more conformal dosimetry and amend for the anisotropy generated by a single line source at the vaginal apex. Differential loading of the channels can also potentially reduce the dose to the bladder and rectum, compared with the single channel cylinder. For example, channels 2 and 5 can be left empty in a 7 channel applicator with 6 peripheral channels, and this will reduce the dose to these organs.

The multichannel applicator minimizes the effect of anisotropy and significantly improves CTV dose coverage at 5 mm from applicator tip by up to 40% (p=0.001). However, it does so by increasing the risk to the vaginal mucosa.

Thus, there is always room for further improvements, and what is needed in the art are improvements in brachytherapy applicator design that allow real-time dose monitoring to allow improved dosimetry, and that also assist in imaging during treatment. It would also be an advantage to accurately measure the source radiation, as afterloaders and other radiation sources typically have fairly high variability, leading to as much as 30% discrepancies between intended and actual source dose.

### BRIEF SUMMARY OF THE INVENTION

This invention combines a solid cylindrical brachytherapy applicator with additional channels (hollow tubes or lumens) provided therein for placement of small diameter scintillation fiber based radiation sensors. The sensors can be used with single channel applicators, but are preferably used with multichannel applicators. Preferably, the sensors are placed centrally and around the periphery, i.e., dividing the applicator into thirds, fourths, fifths, sixths, etc. The channels are preferably placed near to the radiation channels, thus providing accurate dosing information near each source of radioactivity. Thus, the radiation and sensor lumens can be paired, or they can be in triplets, a radiation lumen on each side of a sensor lumen.

We anticipate that in some embodiments, the sensors will be removable and be reused, in which case the applicators and cables can be sold separately. In other embodiments, the applicator is a lightweight plastic applicator sold together with the assembled PSD sensor cables, and the device may be disposable or for single patient use. Where a device is intended for multiple uses, it is typically covered with a disposable plastic sheath for use in patients.

Extremely small diameter sensors are described in U.S. Pat. No. 8,953,912, entitled “Small diameter radiation sensor cable” and incorporated by reference herein in its entirety for all purposes. This patent describes robust and easily made plastic scintillator detectors (PSD) devices have a diameter of 2 mm or less (excluding adaptors). Such a small cable (3 French*) can easily be interested into a channel of about 7 French interior diameter. *The French scale or French gauge system is commonly used to measure the size of a catheter. The French size is three times the diameter in millimeters. A round catheter of 1 French has an external diameter of ⅓ mm, and therefore the diameter of a round catheter in millimeters can be determined by dividing the French size by 3.

Although channels are preferred, as protecting the delicate cable, it may also be possible to have radiation and sensor grooves, wherein the channel is open to the surface of the applicator (see e.g., U.S. Pat. No. 9,132,282, incorporated by reference herein in its entirety for all purposes). However, this may be less preferred as exposing the delicate cables to possible wear. On the other hand, it may be easier to insert reusable cables into a groove.

In preferred embodiments, the diameter of the channel is slightly larger than the diameter of the PSD so that the PSD can be removed and reused. In other preferred embodiments, both the applicator and the external jacket of the PSD are of a smooth material with low coefficient of friction. Preferably, a material with low coefficient of friction (ASTM D3702), or the materials are coated with a low tack coating are used, thus facilitating insertion.

If the friction is high, it can be difficult to insert the PSD into the hollow channel. Thus, the friction should be low, as assessed by ease of repeated insertion (3X) of the PSD into the channel. If the PSD jacket or the interior of the channel in the applicator has high friction, such insertion is difficult, one or the other or both can be modified to reduce friction, or the channel size can be increased to accommodate. For example, the jacket and/or applicators can be coated with an anti-tack coating, or the jacket and/or applicators can be formulated with an anti-tack additive. Talc and glyceryl monostearate (GMS) are known to reduce the tackiness of the films significantly when tested by the method of Wesseling (1999). Silicones are also used for this purpose, as is PTFE powder. In yet other embodiments, the sensor cable is glued into a channel or groove.

In more detail, the invention includes any one or more of the following embodiment(s) in any combination(s) thereof:

A brachytherapy applicator device, comprising:
a) a solid tubular body having a rounded distal end;
b) said solid tubular body having a plurality of hollow radiation lumens
sized to receive an afterloader cable or a radiation source cable;
c) said solid tubular body having a plurality of hollow sensor lumens
sized to receive plastic scintillator detector cables;
d) each sensor lumen being adjacent one or two radiation lumens to
provide one or more lumen pairs or lumen triplets.
A brachytherapy applicator and radiation sensor device, comprising:
e) a solid tubular body having a rounded distal end and a proximal end;
f) said solid tubular body having a plurality of pairs or triplets of
hollow lumens therein, each lumen opening to an exterior at said proximal
end and reaching or nearly reaching said distal end;
g) each pair or triplet of lumens comprising a first radiation lumen sized
to receive an afterloader or a radiation source and second or third sensor
lumens sized to receive plastic scintillator detector cables, said first
radiation lumen less than 3 mm from said second sensor lumen.
A brachytherapy applicator and sensor device, comprising:
h) a solid tubular body having a rounded distal end;
i) said solid tubular body having one or more hollow radiation lumens
sized to receive an afterloader or a radiation source;
j) said solid tubular body having one or more hollow sensor lumens sized
to receive an plastic scintillator detector cable;
k) each of said sensor lumens having a plastic scintillator detector
(PSD) sensor cable therein for measuring radiation dosage.
A brachytherapy applicator and radiation sensor device, comprising:
l) a solid tubular body having a rounded distal end;
m) said solid tubular body having a central hollow radiation lumen sized
to receive an afterloader or a radiation source within 2 mm of a central
hollow sensor lumen sized to receive a plastic scintillator detector cable;
n) said solid tubular body having one or more peripheral radiation lumens
or radiation grooves sized to receive an afterloader or a radiation source,
each within 2 mm of a peripheral sensor lumen or sensor groove sized to
receive an PSD sensor cable.
A device as herein described, made of plastic.
A device as herein described, made of plastic by injection molding a
semispherical distal end and a tubular proximal end, said distal end and
said proximal end operably coupled together.
A device as herein described, each second or third sensor lumen
comprising a plastic scintillator detector sensor cable for measuring
real-time radiation.
A device as herein described, each sensor lumen being within 3 mm, 2
mm or 1 mm of a nearest adjacent radiation lumen. The lumens can be
arranged in pairs or triplets.
A device as herein described, composed of at least a nose cone component
comprising said rounded distal end and a main body component, said nose
cone and said main body being operably connected.
A device as herein described, said nose cone comprising an inner nose
cone with outer grooves on an exterior surface thereof, and an outer
nose cone with inner grooves on an interior surface thereof, said
outer grooves aligning with said inner grooves on assembly to form
said lumens. Other components can include locking stoppers for
controlling depth of applicator, and locking devices for controlling
depth of source and sensors.
A device as herein described, further comprising a inserter tube component
that fits to a proximal end of said main body, said inserter tube having a
hollow interior for holding a plurality of cables.
A device as herein described, each component having a male or female
end for alignment with an female or male end of an adjacent component
on assembly.
A device as herein described, wherein said male or female ends are
arranged asymmetrically.
A device as herein described, each component made of polystyrene,
polycarbonate/ABS Alloy, PEI, polysulfone, PEEK, acetal or high impact
polystyrene nylon.
A device as herein described, wherein said sensor lumens are larger in
diameter than said radiation lumens. Alternatively, said sensor lumens
are larger in diameter than said radiation lumens, except for a larger
central radiation lumen.
A device as herein described, wherein said solid tubular body is made of at
least two components: i) a semispherical head portion operably connected
to ii) a cylindrical body portion.
A device as herein described, said device further including a semi-
compliant or non-compliant balloon surrounding a distal end of said solid
tubular body.
A device as herein described, wherein said sensor lumens are located
within 2 mm of said radiation lumens, providing pairs of lumens.
Alternatively, said sensor lumens are located within 2 mm of a pair
of radiation lumens such that said pair of radiation lumens bracket
each sensor lumen, providing a triplet of lumens.
A device as herein described, each of said PSD sensor cables having a
radio-opaque marker thereon, preferably on a distal tip.
A device as herein described, said PSD sensor cables being removably
inserted from said sensor lumens, or glued thereinto.
A device as herein described, each of said PSD sensor cables comprising
an opaque jacket enclosing a plastic scintillating fiber at a distal tip
directly abutting a fiber optic cable, said fiber optic cable longer
than said solid tubular body and terminating in an adaptor for reversible
connection to a separate photodetector measuring device.
A device as herein described, each of said PSD sensor cables having a
diameter of 3 mm or less, or 2 mm or less.
A device as herein described, said lumens having a smooth low friction
surface, preferably having coefficient of friction of <0.3 or <0.2.
A brachytherapy treatment method comprising:
inserting the device into a body cavity having a tumor;
inserting PSD sensor cables into each of said sensor lumens (unless
already present);
connecting each of said PSD sensor cables to a photodetector measuring
device;
inserting an afterloader containing a radiation source into one or more
of said radiation lumens;
treating said tumor and measuring dosage during said treatment;
measuring a dose of said radiation source;
retracting said afterloader;
disconnecting said PSD sensor cables from said photodetector measuring
device; and removing said device from said cavity.
Another treatment method comprises:
inserting the device into a cavity having a tumor;
imaging said radio-opaque markers and repositioning said device as
needed to target a first treatment site on or in said tumor;
connecting each of said PSD sensor cables to a photodetector measuring
device (unless already present);
inserting an afterloader containing a radiation source into one or more
of said radiation lumens;
treating said tumor and measuring dosage during said treatment;
measuring a dose of said radiation source;
optionally repeating steps b-f for a second or more treatment sites;
retracting said afterloader;
disconnecting said PSD from said photodetector measuring device; and
removing said device from said cavity.
A method as herein described, wherein a depth of said sensor in said
sensor lumen is adjusted to a desired level near a depth of said radiation
source.

### BRIEF DESCRIPTION OF THE DRAWINGS

A better understanding of the present invention can be obtained with the following detailed descriptions of the various disclosed embodiments in the drawings:

FIG. 1. A prior art multichannel brachytherapy applicator. The Capri 13 channel applicator by Varian consists of a single central catheter CH1, an inner array of six catheters CH2, and an outer array of six catheters CH3. A balloon B1 covers the distal end in an airtight manner, and its inflation via valve V1 allows vaginal tissue to be pushed away from the radiation source, allowing targeting of e.g., the cervix. The lumens L1 in the Capri are impregnated with a barium sulfate to allow simple catheter visualization. Cover C1—here shown separately—covers the lumens, presenting a smooth exterior and allowing accurate depth placement via the depth markings.

FIG. 2. Another prior art brachytherapy applicator with solid tubular shape. The distal head portion H2 and body portion P2 are shown separate and the head portion H1 is cut away to allow the channels CH2 to be visible, as well as lumens L2. An exterior stopper S1 allows the technician to accurately and reproducibly place the depth of the device into the body cavity.

FIG. 3 Perspective view of multichannel brachytherapy applicator with PSD sensors.

FIG. 4 Side view of the device of FIG. 3.

FIG. 5 Cross section of the device of FIG. 3.

FIG. 6 Dosing graphic.

FIG. 7 Method of use.

FIG. 8A-D Small diameter sensor cable. FIG. 8A Complete duplex sensor cable, FIG. 8B enlargement of detector end of duplex cable, FIG. 8C enlargement of adaptor end of duplex cable. FIG. 8D Simplex cable.

FIG. 9A-D small diameter sensor cable. FIG. 9A enlargement of detector end of duplex cable. FIG. 9B complete duplex sensor cable. FIG. 9C enlargement of adaptor end of duplex cable. FIG. 9D cross-section through line D-D in FIG. 9C.

FIG. 10. Plastic selection for use in injection molding.

FIG. 11. Exemplary coefficient of friction for some plastics.

### DETAILED DESCRIPTION OF THE INVENTION

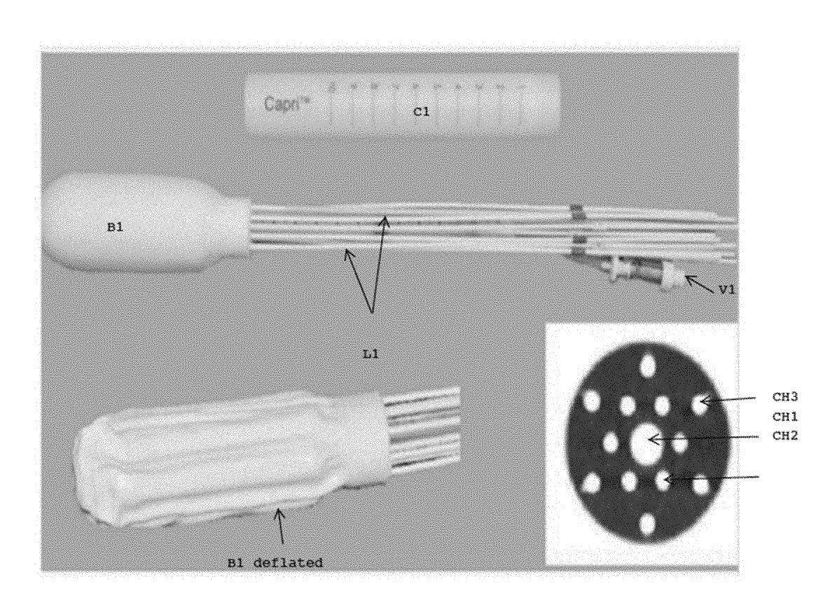

A prior art multichannel brachytherapy applicator “Capri” by Varian is shown in FIG. 1. The Capri 13 channel applicator consists of a single large central catheter (CH1), an inner array of six smaller catheters (CH2), and an outer array of six smaller catheters (CH3). This orientation is clearly visible in the cross section. A balloon B1 covers the distal end in an airtight manner, and its inflation via valve V1 allows vaginal tissue to be pushed away from the radiation sources or lumens, allowing targeting of e.g., the cervix. The lumens L1 of the Capri are impregnated with a barium sulfate to allow catheter visualization. Cover C1 covers the lumens when assembled, presenting a smooth exterior and allowing accurate depth placement via the depth markings.

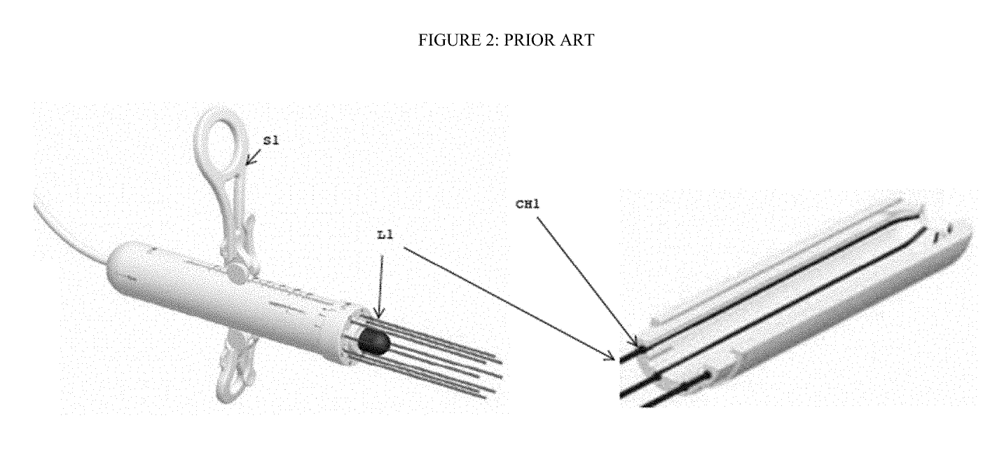

FIG. 2 shows another prior art vaginal brachytherapy applicator with solid tubular shape. The distal head portion H2 and body portion P2 are shown separated and the head portion H2 is also partially cut away to allow the channels CH2 to be visible, as well as lumens L2 into which the radioactive material is placed.

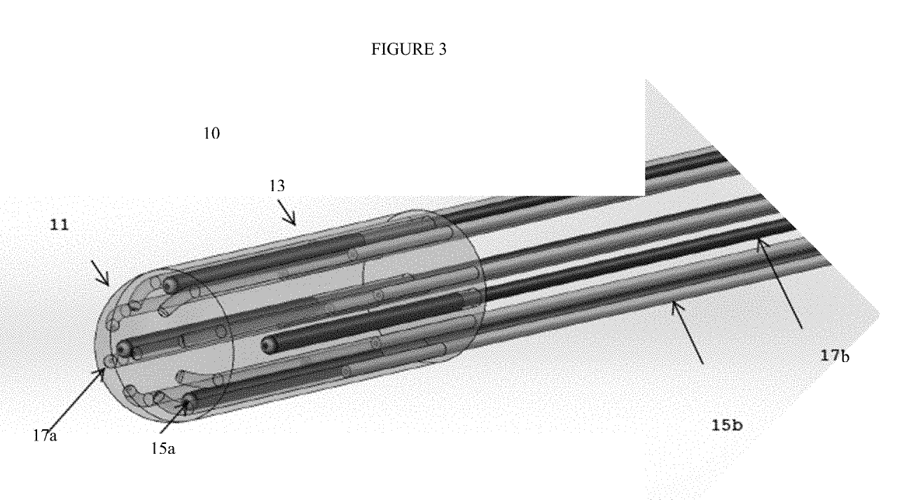

FIG. 3 shows a perspective view of one embodiment of the brachytherapy applicator with PSD sensor cables. The applicator 10 has a head portion 11 and body portion 13, both of which are solid except for channels 15a and 17a, into which the radiation lumens 15b and PSD sensor cables 17b fit. In this figure, the radiation lumens, 15b are not pushed all the way to the end of the channels 15, but they would be during patient usage. Alternatively, depth can be varied as part of the treatment plan, depending on tumor location.

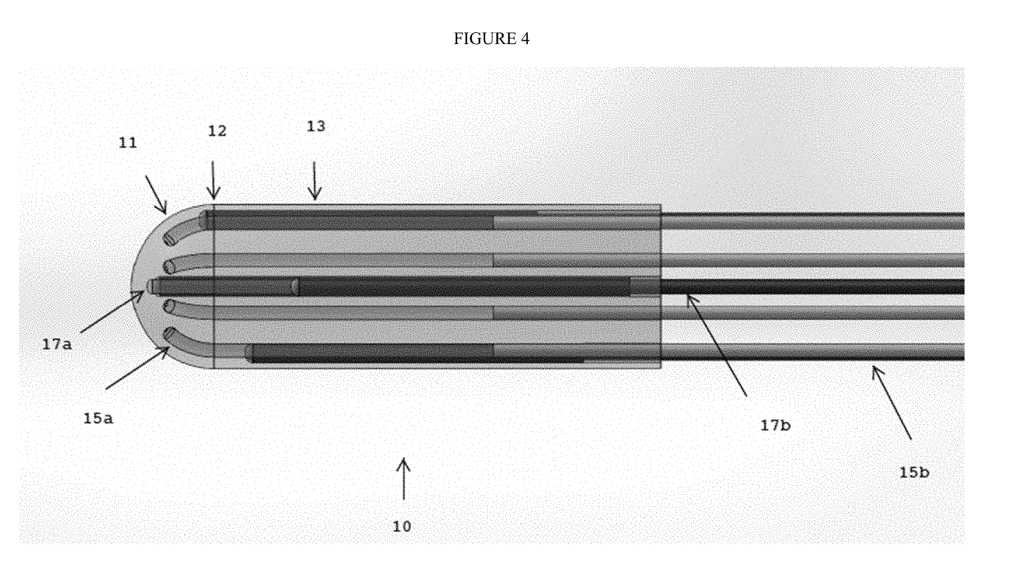

The same device is shown in side view in FIG. 4, where join line 12 where the two parts join is clearly visible.

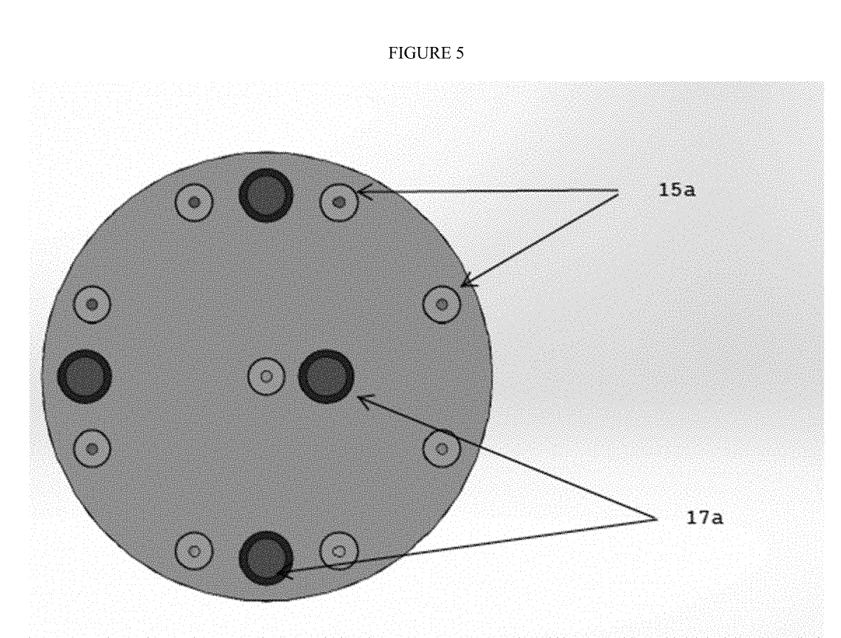

A cross section at line 12 is shown in FIG. 5, and it can be seen that the channels 17a are very near channels 15a. Ideally, the two channel pair would be about 0.5-3 mm or about 1-2 mm of each other, but not so close that structural integrity of the channel is compromised. Thus allows accurate dosimetry near each lumen, allowing better control of IR and TV, and closer to ideal PTV coverage.

Any method of manufacture can be used, including one or more of molding, drilling, laser cutting, 3D printing, injection molding, insert molding, gas assisted injection molding, multicolor injection molding, outsert molding, push-pull injection molding, reaction injection molding, sandwich injection molding, thermoforming or vacuum forming, autoclave molding, matrix injection, filament winding, hand lay-up, hot pressing, composites, pultrusion, and the like.

In one embodiment, the semispherical head 11 and solid tubular body 13 are made as separate, high fidelity pieces by injection molding and then bonded together by adhesive, welding, heat, and the like, making sure the channels are correctly aligned. A notch and protrusion (not shown) can simplify the alignment process. This method has the potential to make the applicators so inexpensive as to be considered disposable, thus negating the need for a sterile plastic cover in use. The PSD sensor cables may also be disposable, but at the moment it is contemplated that they will be reused, since the adaptor is not inexpensive.

The brachytherapy applicator and PSD sensor can also be used with a balloon. For example, U.S. Pat. No. 7,678,040 describes separate vaginal and prostate balloons that can be used with the brachytherapy applicators. U.S. Pat. No. 7,727,137, U.S. Pat. No. 7,918,778, and U.S. Pat. No. 7,678,040 also describe brachytherapy applicators with integral balloons. Each of these patents is incorporated by reference herein in its entirety for all purposes.

Two basic types of balloons are used in the medical industry. One is the high-pressure, non-elastic, dilatation or angioplasty-type balloon used to apply force. The other is the low-pressure, elastomeric balloon typically made of latex or silicone that is used primarily in fixation and occlusion. High-pressure balloons are molded to their inflated geometry from “non-compliant” or “low-compliant” materials that retain their designed size and shape even under high pressure. They are thin-walled and exhibit high tensile strength with relatively low elongation. Low-pressure balloons are typically dip-molded in a tubular shape which is then expanded several times its original size in use, thus these balloons cannot be inflated to precise dimensions or retain well defined shapes and high pressures.

In one embodiment, the balloon is a simple blow molded, dip molded, or cold molded unitary balloon with no edges and no edge welding. Such balloon has advantage as being simple to make, and less subject to leakage at welds, since the only weld is the proximal weld to the brachytherapy applicator. However, the best material for such a balloon is not elastic, thus providing a non-compliant surface, or at least only a semi-compliant material is used.

Crosslinked polyethylene (PE) and polyester polyethylene terephthalate (PET) have been adopted for high-pressure balloons. Nylon, while not as strong as PET or as compliant as PE, was seen as a compromise because it was softer than PET, but relatively thin and relatively strong. Today most high-pressure medical balloons are made from either PET or nylon. PET offers advantages in tensile strength, and maximum pressure rating while nylon is softer. See Table 1 for a comparison of various high-pressure balloon materials.

TABLE 1
Comparison of High-Pressure Balloons Made with Various Materials

| | | | | | Max. Rated | | |
|---|---|---|---|---|---|---|---|
| | | | | | Pressure for | | |
| | Tensile | | | | PTCA* | | Sterilization |
| Materials | Strength | Compliance | Stiffness | Profile | ATM | PSI | Methods |
| PET | High-Very | Low- | High | Low | 20 | 294 | EtO |
| | High | Medium | | | | | or |
| | | | | | | | Radiation |
| Nylons | Medium- | Medium | Medium | Low- | 16 | 235 | EtO |
| | High | | | Medium | | | |
| PE | Low | High | Low | High | 10 | 147 | EtO |
| (crosslinked) | | | | | | | or |
| and other | | | | | | | Radiation |
| polyolefins | | | | | | | |
| Polyurethanes | Low- | Medium- | Low- | Medium- | 10 | 147 | EtO |
| | Medium | High | Medium | High | | | |
| PVC (flexible) | Low | High | Low | High | 6-8 | 88-117 | Radiation |

*The maximum rated pressure is based on practical limitations and usefulness. Obviously, very thick walls can be used with any material to increase the rated pressure; however, the balloon would be useless.

The balloon can be a separate device that fits over the applicator, or can be a part of the applicator, as desired. Examples of both types are available in this literature.

The balloon itself is sized and shaped for the cavity in question, and preferably provides equidistant spacing for the tissue at most if not all points of the balloon. As noted above, the simplest way to do this is with a non-compliant or semi-compliant material and carefully design of balloon shape and size.

However, other methods of shaping the balloon are also possible. A balloon can be made flat for example with the use of internal welds to an opposite surface or middle layer, or small connectors connecting one side to the other. Examples are a toirodal balloon (U.S. Pat. No. 9,227,084) or dual nested (concentric) balloon shape (U.S. Pat. No. 9,283,402), wherein the outer surface can be controlled with respect to the inner surface. Each of these patents is incorporated by reference herein in its entirety for all purposes.

The brachytherapy applicator with PSD sensor cables can also comprise radio-opaque markers that can be used in imaging for accurate placement and imaging. Opaque markers can be letters indicating top (T) or right (R) and left (L) sides, or numbers or any other shape, and can be particularly advantageous for those devices whose shape is not radially symmetrical. A small marker (a dot) can also be placed on the very tip of the PSD sensor to allow the user to accurately position the PSD sensor with respect to the target tissue.

As another option, the PSD jacket material or cap material can include a radiopaque filler, thus making the sensor end of the sensor cable visible. It may be necessary to use different markings for the PSD sensor cable so that they can be easily differentiated from the radiation lumens. For example, the cap housing of the plastic scintillator can be impregnated with radiopaque filler, whereas the radiation lumens are impregnated throughout, or a distal tip marker will suffice as well to distinguish the other lumens. In other embodiments, the PSD cable can be printed with concentric rings or lines or some other pattern distinguishable from the radiation lumens.

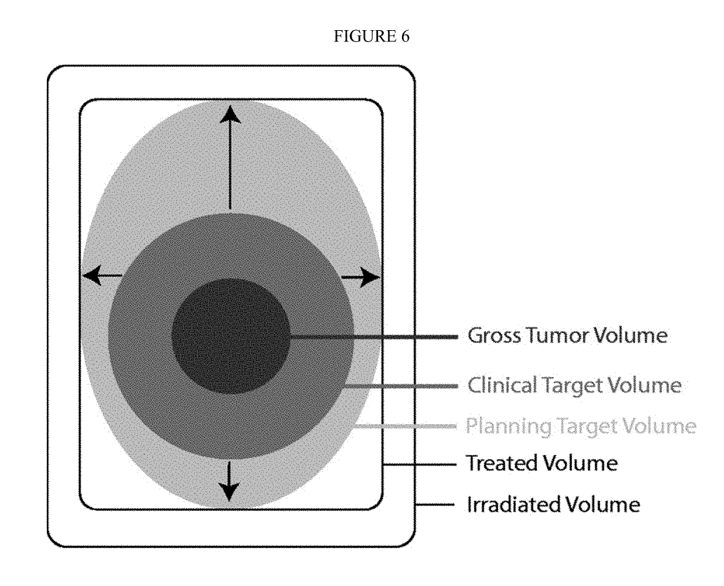

In order to accurately plan the brachytherapy procedure, a thorough clinical examination is performed to understand the characteristics of the tumor. The gross tumor volume (GTV) is determined by imaging and clinical target volume (CTV), planned treatment volume (PTV), and organs-at-risk (OAR) are delineated (FIG. 6).

A range of imaging modalities can be used to visualize the shape and size of the tumor and its relation to surrounding tissues and organs. These include x-ray radiography, ultrasound, computed axial tomography (CT or CAT) scans and magnetic resonance imaging (MRI), and the like. The data from many of these sources can be used to create a 3D visualization of the tumor and the surrounding tissues.

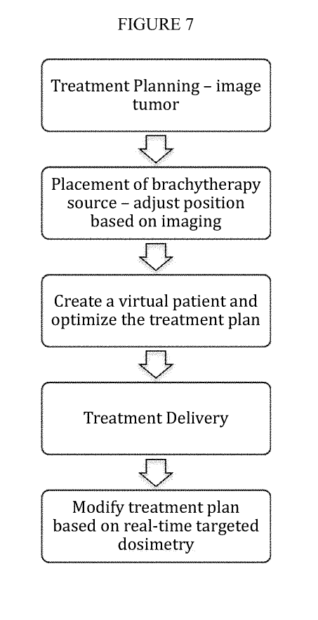

Using this information, a plan of the optimal distribution of the radiation sources can be developed (FIG. 7). This includes consideration of how the source carriers (applicators), which are used to deliver the radiation to the treatment site, should be placed and positioned. Applicators are non-radioactive and are as described herein, having with at least one lumen sized to accept the afterloader, and at least one lumen sized to fit a PSD sensor cable. This initial planning helps to ensure that ‘cold spots’ (too little irradiation) and ‘hot spots’ (too much irradiation) are avoided during treatment, as these can respectively result in treatment failure and side-effects. It also helps to reduce dosage to the OAR.

Before radioactive sources can be delivered to the tumor site, the applicators have to be loaded with the PSD sensors, unless they are sold as a combined unit. The assembled brachytherapy applicator with PSD sensor cables is inserted into the body cavity, the balloon (if any) inflated, and the device positioning confirmed by imaging, such that the device is correctly positioned in line with the initial planning. Imaging techniques, such as x-ray, fluoroscopy and ultrasound are typically used to help guide the placement of the device to the correct position and to further refine the treatment plan.

Once the brachytherapy applicator plus PSD sensors are inserted, and positioning confirmed, the handle e.g., can be held in place against the skin using sutures or adhesive tape or clamp to prevent them from moving. If desired, further imaging can be performed to guide detailed treatment planning.

The images of the patient with the applicators in situ are imported into treatment planning software. The treatment planning software enables multiple 2D images of the treatment site to be translated into a 3D ‘virtual patient’, within which the position of the applicators can be defined. The spatial relationships between the applicators, the treatment site and the surrounding healthy tissues within this ‘virtual patient’ are a copy of the relationships in the actual patient.

To identify the optimal spatial and temporal distribution of radiation sources, the treatment planning software allows virtual radiation sources to be placed within the virtual patient. The software shows a graphical representation of the distribution of the irradiation. This serves as a guide for the brachytherapy team to refine the distribution of the sources and provide a treatment plan that is optimally tailored to the anatomy of each patient before actual delivery of the irradiation begins. This approach is sometimes called ‘dose-painting’. Herein, dose painting can be greatly improved with real-time feedback about delivered radiation. The sensor cables can also provide dose information about the source.

The radiation sources used for brachytherapy are always enclosed within a non-radioactive capsule. The sources can be delivered manually, but are more commonly delivered through a technique known as ‘afterloading’. Afterloading involves the accurate positioning of non-radioactive steerable applicator adjacent or in the treatment site, which are subsequently loaded with the radiation sources. In manual afterloading, the source is delivered into the applicator by the operator.

Remote afterloading systems are preferred as they provide protection from radiation exposure to healthcare professionals by securing the radiation source in a shielded safe. Once the applicators are correctly positioned in the patient, they are connected to an ‘afterloader’ machine (containing the radioactive sources) through a series of connecting guide tubes. The treatment plan is sent to the afterloader, which then controls the delivery of the sources along the guide tubes into the pre-specified positions within the applicator. This process is only engaged once staff is removed from the treatment room. The sources remain in place for a pre-specified length of time, again following the treatment plan, following which they are returned along the tubes to the afterloader. With the device of the invention, the guide tubes may not be needed, as they source wires can insert directly into the lumens of the applicator.

At some point, the sensor cables have to be connected to a photodetector system for real-time measurement of the dose. This can be done at any point in the procedure, but it is likely that the optimal time will be after accurate positioning and before connecting to the afterloader.

Once the afterloader is connected, treatment can commence, and dosimetry can be measured on a real-time basis at targeted locations via the PSD sensors within the applicator. Adjustments to positioning and/or total dosage or delivery rates can be made based on this real-time feedback, and the adjustments can be applied immediately, or in the next treatment session, as appropriate. Once the desired dosage level is reached for a given treatment session, the treatment is stopped, and the user can then reposition the applicator for a second target site (if any). This can be repeated as often as necessary to target the tumor.

On completion of delivery of the radiation, the devices are disconnected from the afterloader and photodetector. The balloon (if any) is deflated, and the device is carefully removed from the body. Patients typically recover quickly from the brachytherapy procedure, enabling it to often be performed on an outpatient basis.

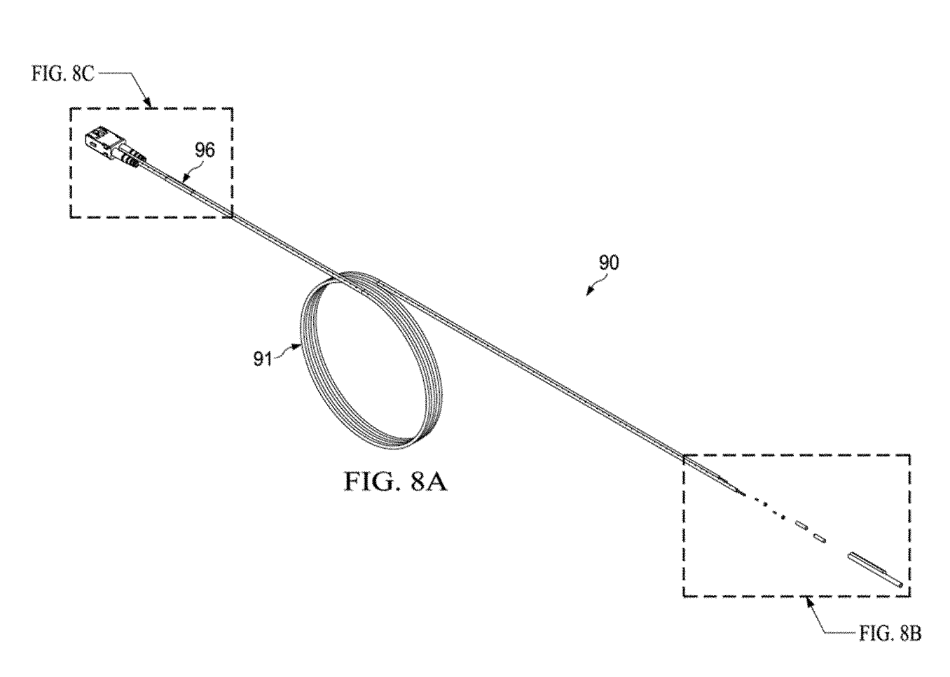

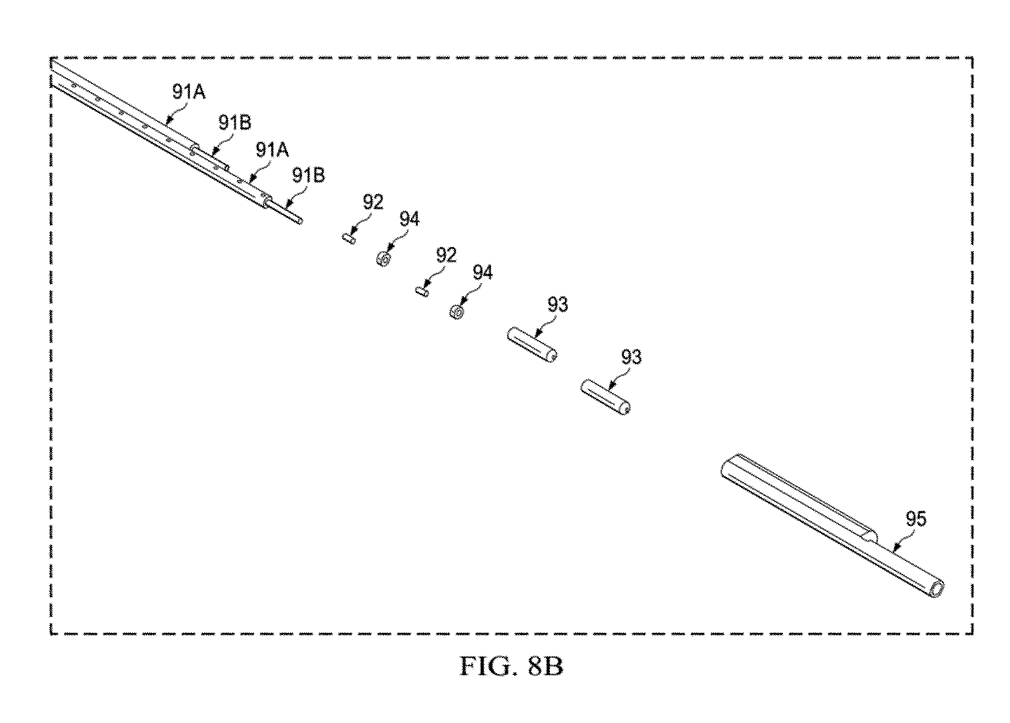

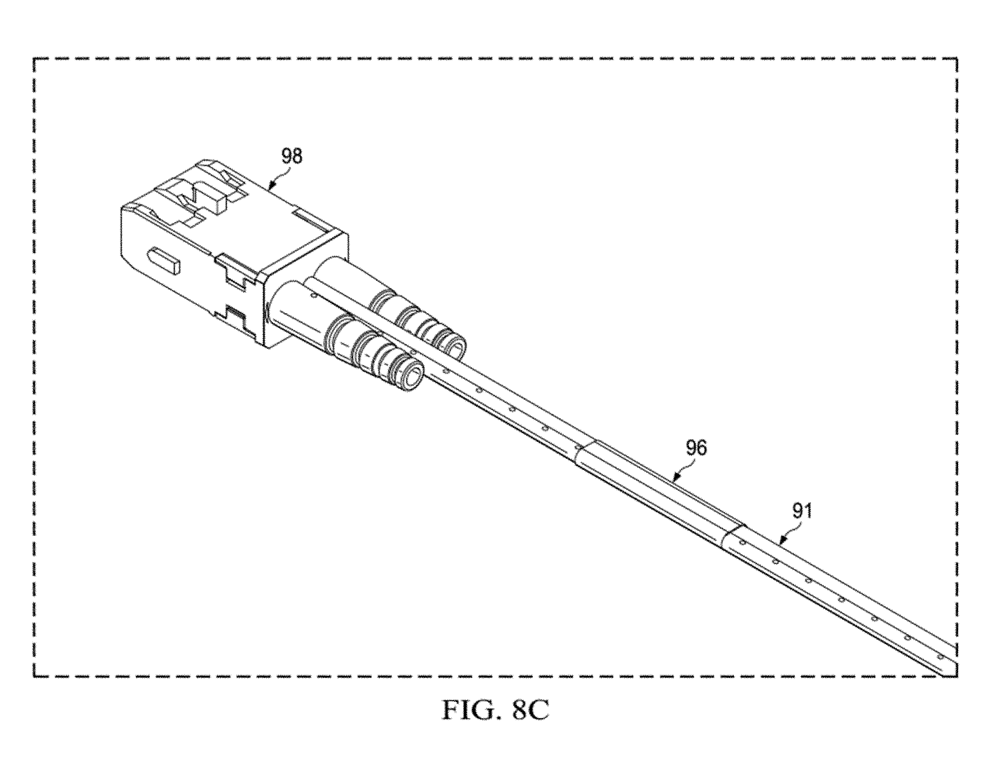

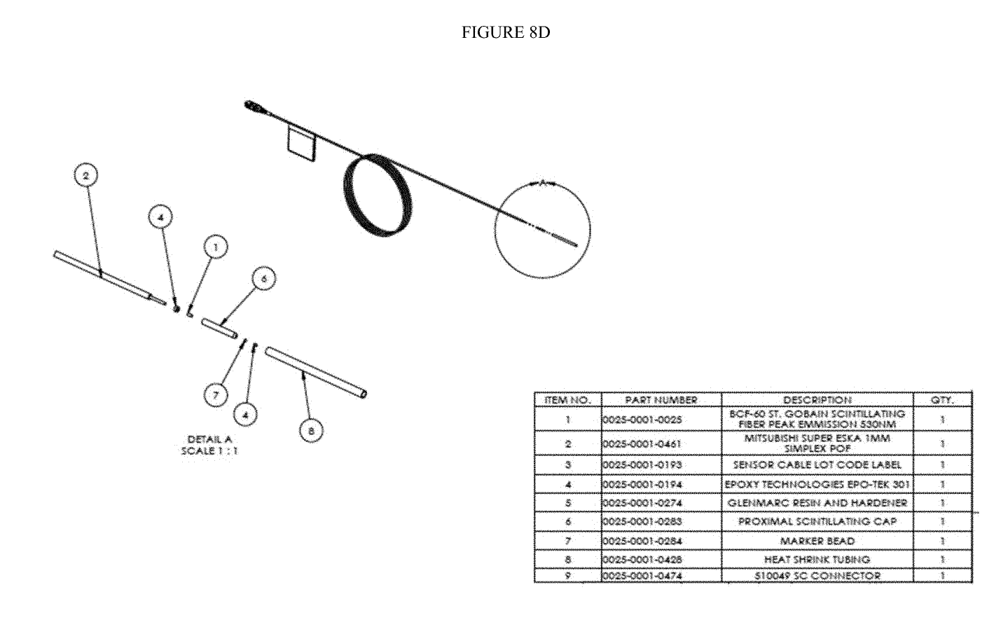

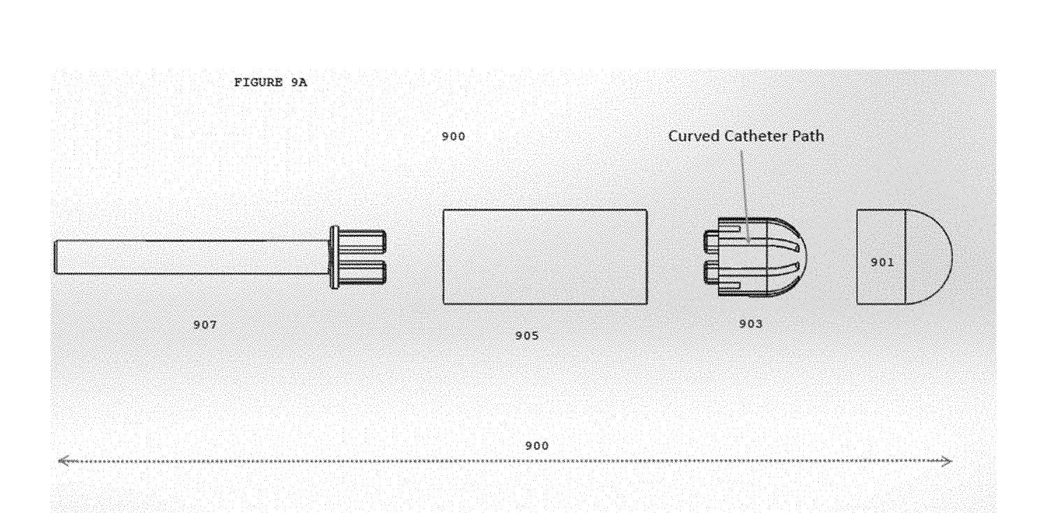

Plastic scintillator based dosimeters are described in our prior patents and one embodiment is shown in FIG. 8A-D. FIG. 8A shows one embodiment of an assembled radiation sensor 90, while FIG. 8B shows the exploded sensor components and FIG. 8C shows the connector 98 at the proximal end of the cable. The device and cap is described in more detail US20120281945.

In FIG. 8B, a duplex scintillation detector cable 90 has a first and second optical fibers 91, but the same principals can be used for varying number of sensors. We anticipate that single PSD sensor units will be used herein due to size limitations, but variations are possible if the lumens are increased in size.

The jacket or covering 91A has been stripped or removed from the portion of the first optical fiber 91 adjacent to the distal ends of each fiber, leaving a portion of each optical fiber 91B exposed. First and second scintillating fibers 92 are shown, along with drop of adhesive 94 and fiber cap 93. The length of scintillating fibers 92 can be varied, according to needed sensitivity and size of area to be assessed, but typically 1-10 mm or 2-3 mm of length will suffice.

The scintillating fibers 92 fit into the fiber caps 93, followed by the naked optic fibers 91B, and a drop of epoxy 94 on the sides (not ends). Heat shrink tubing 95 covers the components. At the far end, an adaptor 98 is found, in this case a dual jack adaptor. Label 96 is also shown, but may be placed anywhere on the cable or even on packaging and is not considered material. There is no adhesive 94 on the abutted ends or faces of the respective scintillating fibers 92 and optical fibers 91, thus signal are reliability are both optimized.

The duplex optical fiber 91 may be a Super Eska 1 mm duplex plastic optical fiber SH4002 available from Mitsubishi Rayon Co., Ltd. of Tokyo, Japan, although other duplex optical fibers are also contemplated. Although duplex optical fibers 91 are shown, it is also contemplated that a single optical fiber may be used or additional fibers can be added.

The scintillating fibers 92 may be a BCF-60 scintillating fiber peak emission 530 NM available from SAINT-GOBAIN CERAMICS & PLASTICS™, Inc. of Hiram, Ohio, although other scintillating fibers are also contemplated.

A simplex radiation sensor cable is shown in FIG. 8D, wherein the numbers are provided in the adjacent legend.

The brachytherapy applicator could also comprise passive radiation sensors, such as is used in radiation badges, but these are less preferred as not offering real-time information. Nevertheless, they may be advantageous in certain circumstances. Electronic radiation sensors can also be used, but will contribute significantly to expense, and are expected to be less appropriate at this time. Thus, the small PSD sensor is currently preferred.

In one manufacturing method, the main body is made from a plastic extrusion process, while the inserter and nose-pieces are plastic injection molded. Alternatively the main body can be made as a solid piece and the straight lumens drilled or lasered out. As yet another alternative, the device can be 3D printed.

In one embodiment, the nose-piece is made of an inner and outer shell in order to mold curved paths for the catheter tubes. The inner and outer nose-pieces are keyed together to guarantee alignment and can be bonded, glued, plastic welded or snap fit together.

The nose assembly and inserter both contain lock and key alignment features which allow accurate alignment with the channels of the main body. The pieces can then be bonded, glued, plastic welded or snap fit together. The extrusion process for the main body allows the cost to be contained to that of a disposable device and also allows for simple and inexpensive changes to the overall diameter and length. Another advantage of the extrusion process is the accuracy of the channel placement and the surface quality of the catheter and sensor tunnel inner diameters as opposed to creating the channels out of multiple pieces.

In one embodiment, the main body extrusion includes locking connectors (not shown) attached directly to the main body or at the end of a short flexible tube. The locking connectors allow the sensor cables to be locked in place when at the correct depth and then be unlocked to pull out of the device. This allows the sensor cables to be cleaned, disinfected and then reused. The locking connectors can use compression on the jacket of the sensor cable to prevent movement of the cable. This compression can come from a deformable material such as silicone and can be in the form of a collet.

The locking connector can also be a two-part design where one half of the connector is bonded to the sensor cable and a mating connector is attached to the applicator body. These two connectors lock together, preventing any movement of the sensor cable. The connectors can click in place with a spring loaded snap feature.

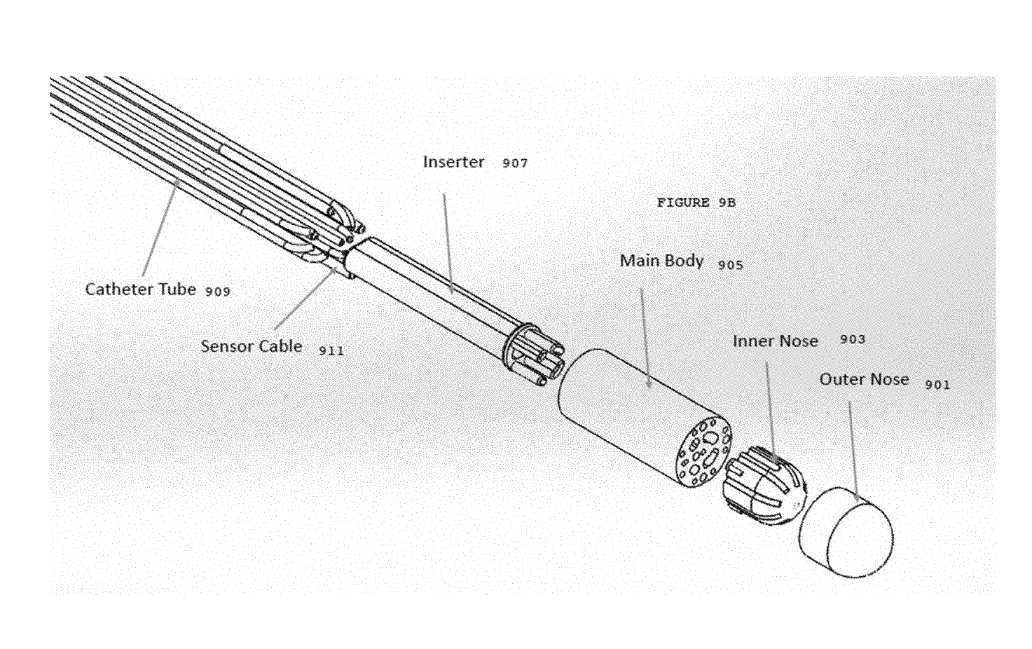

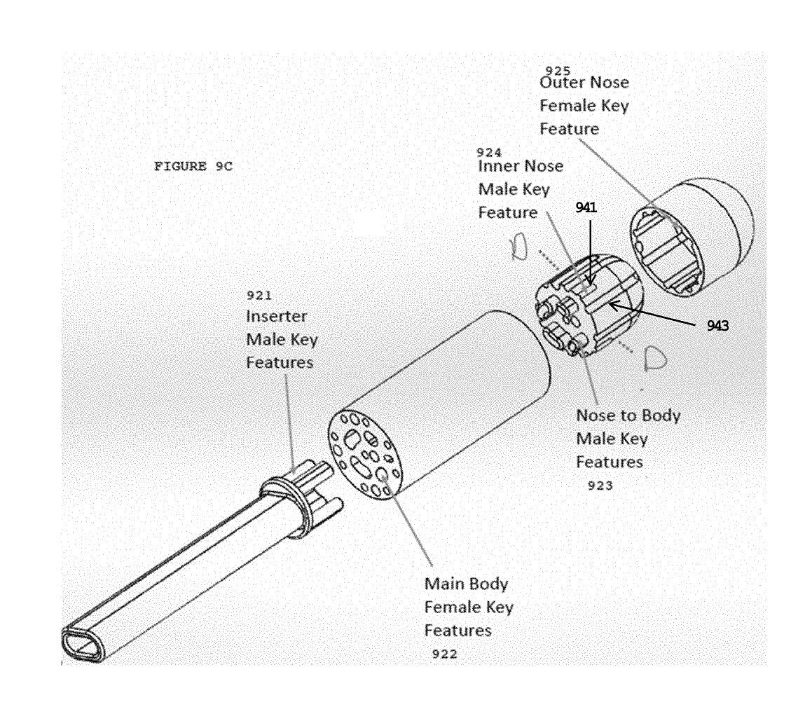

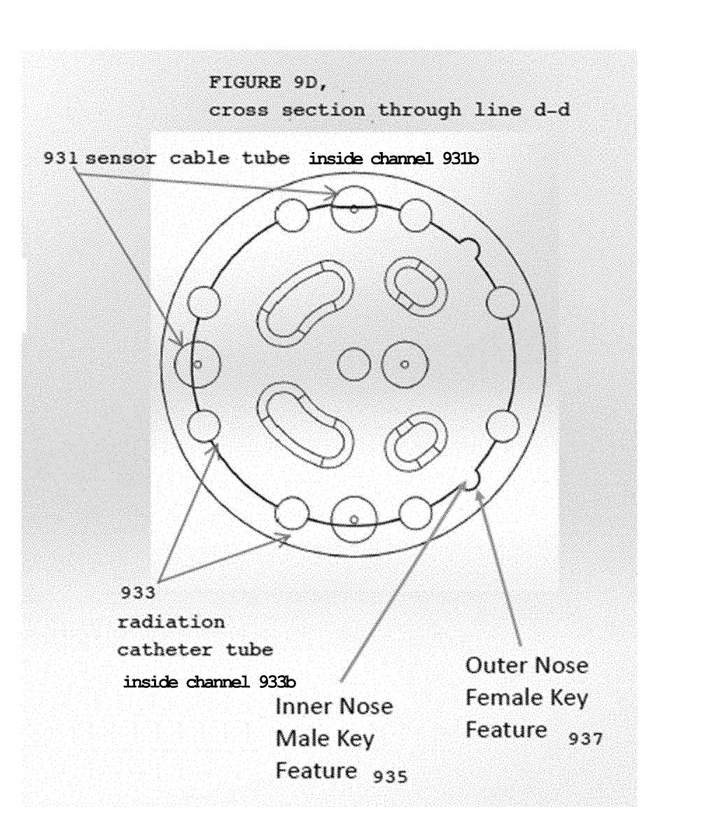

Some of these additional embodiments are seen in FIG. 9A-D. In FIG. 9A, we see the device 900 is composed of four parts that assemble to make the final applicator 900. The outer nose cone 901 provides half of the sensor and radiation channels on inner surface thereof. The outer nose cone 901 fits over the inner nose cone 903, which has the other half of these two channel types on an outer surface thereof. The next part is the main body 905 and inserter 907.

Additional detail is shown in the perspective view of FIG. 9B, where we can see the radiation cables 909 and sensor cables 911. These feed into inserter 907, into the channels in the main body, and then into the channels formed by the assembled nose cone. Obviously the channels must align from part to part, and thus each part has a male protruding element that fits into a female recessed element, ensuring correct alignment in the assembled device 900.

These “lock and key” features are seen in better detail in FIG. 9C, where it is apparent that inserter 907 is generally a hollow tube that all cables can feed into as a bundle. Male protrusions 921 on a distal end of said inserter 907 fit into female recessions 922 on a proximal end of the main body 905. Notice also, that asymmetric lock and key features may be preferred for alignment reasons. Similarly the distal end of main body 905 has female recessions 922 that cannot be seen from this angle. Of course any female and male parts on any component can be reversed, and male and female parts can be mixed on the same component end.

Main body also has channels 931b and 933b that align with the same channels 931 and 933 in the assembled nose cone. In the view in FIG. 9C, it can be seen that the exterior surface of inner nose cone 903 has half channels or grooves 941 and 943, which when assembled with the outer nose cone cooperate with similar grooves 951 and 953 on an interior surface thereof to make the assembled channels 931 and 933, seen in end view FIG. 9D.

When assembled, the grooves provide a passageway for the source wires or catheters containing same, and also for the sensor cables. Since the body is hard, it will be possible to eliminate usage of catheters for the source wires, and directly insert the source wires instead. Further, since the device is made inexpensively with plastic and (preferably) with high precision injection molding, the cost can be low enough to provide a disposable applicator, assuming that sensor cost can be brought low enough. Thus, the device can be sterilized, if desired, used without an outer sheath, and then thrown away.

The inner and outer nose cones also have a lock and key system to ensure correct assembly and alignment. Thus, one or more male features 924 on the inner nose cone 905 fits into corresponding female features 925 on outer nose cone 901. The asymmetry of the male connectors 923 is clearly visible in the end view of the assembled nose cone at FIG. 9D. Also visible is the way that the various grooves (941, 951, 943, 953) align to create the channels (931, 933) at the interface (line) between the inner and out nose cone components. Also visible is the arrangement of sensor channels to radiation channels, in this case two radiation channels bracket a sensor channel. Thus, when fully assembled, a single sensor cable can detect radiation from two radiation channels, but other arrangements are possible.

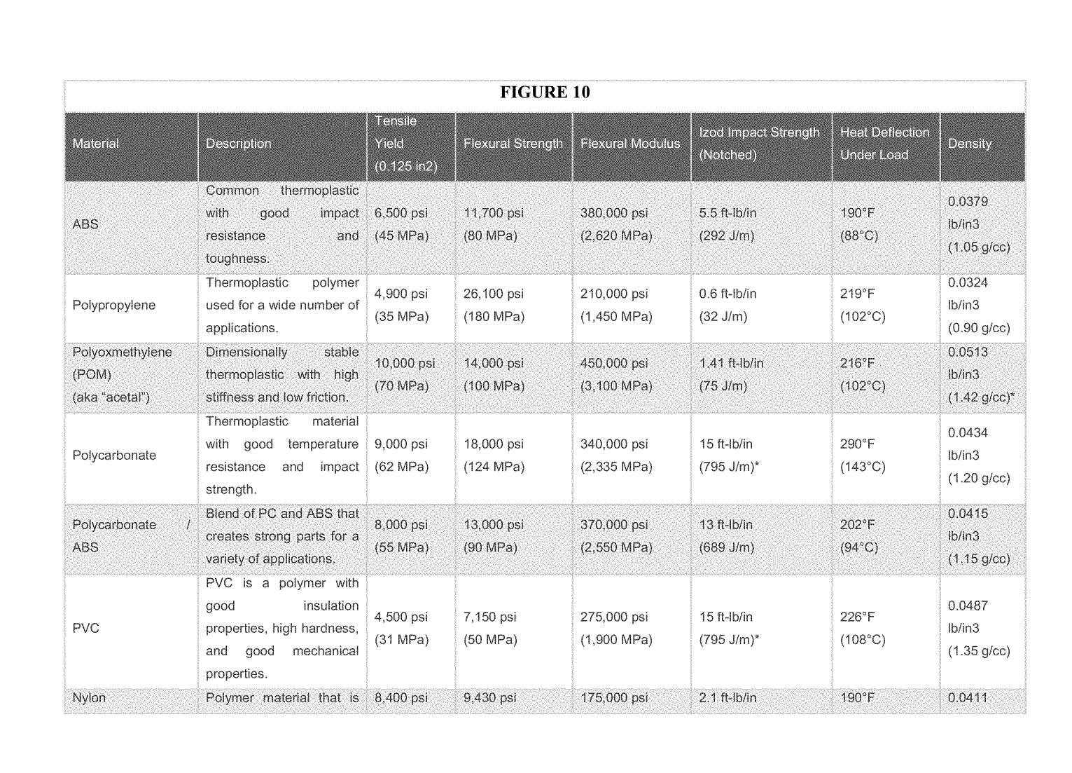

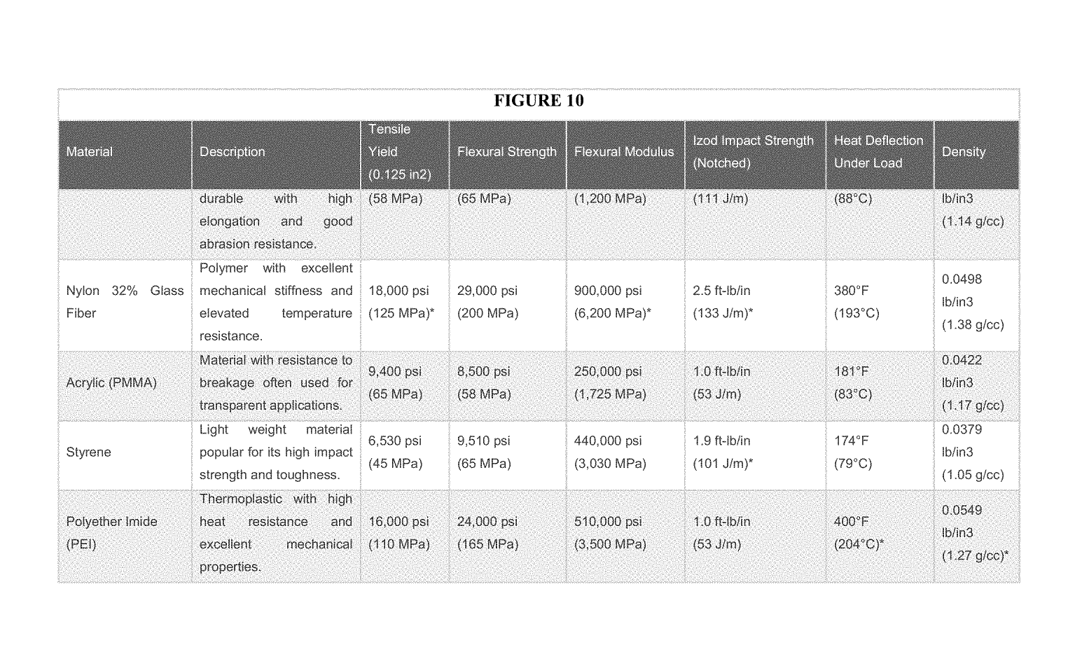

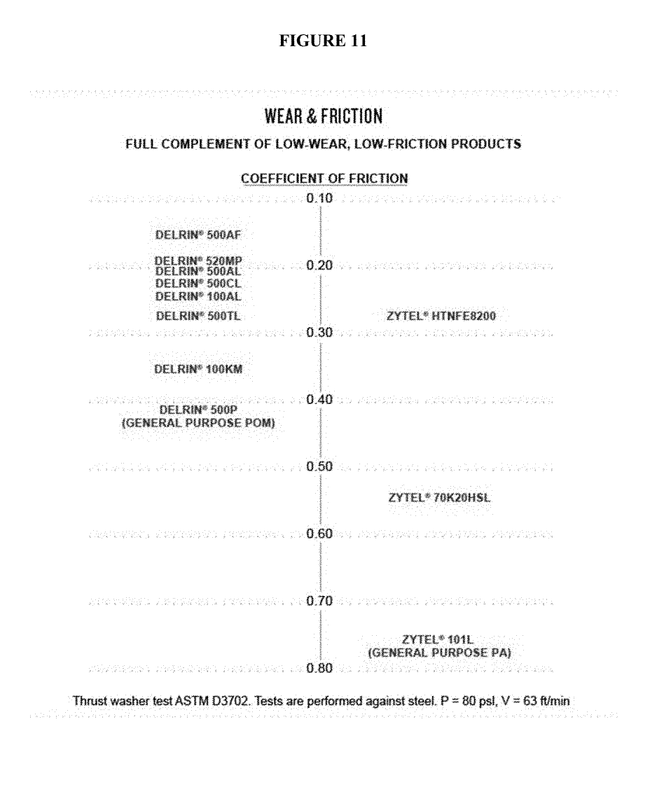

There are many materials suitable for use in injection molding, but some preferred materials are polystyrene, polycarbonate/ABS Alloy, PEI, polysulfone, PEEK, acetal (e.g. polyoxymethylene) and high impact polystyrene. Other suitable materials are described in FIG. 10. Exemplary low friction materials are shown in FIG. 11.

The term “distal” as used herein is the end of the device inserted into the body cavity, while “proximal” is opposite thereto and is closest to the medical practitioner deploying the device. The terms top and bottom are in reference to the figures only, and do not necessarily imply an orientation on usage. The length of applicator plus handle and cables is the longitudinal axis, while a horizontal axis and vertical axis cross the longitudinal axis, and the cross sections are shown across the longitudinal axis.

As used herein a “solid tubular body” refers to a cylindrical body that is not hollow, although it may have a few small lumens drilled thereinto of small volume (<10%). This is contrasted with a hollow tubular body, which has a large central hollow space that occupies at least 50% of the cylinder volume. It is also contrasted with a “partial tubular body,” which is only a section of tube that has been sectioned along its long axis (e.g. half a cylinder or “semicylinder”).

As used herein, a “channel” is completely enclosed by the solid body, and will typically travel from at or near the distal tip to the proximal end of the applicator—the proximal end being open to allow insertion. Channels can be formed by matching or aligning a pair of grooves.

As used herein a “groove” is on the surface of the applicator, or an unassembled portion or component thereof, such that the groove opens to the surface of the applicator or component.

As used herein, a “low-compliance” balloon will expand <10% when inflated to the rated pressure, and preferably <5%. A high-compliance balloon will stretch >18%. A “semi-compliant” balloon will stretch between 10-18%, but preferably between 10-15%.

As used herein the “GTV” or gross tumor volume is what can be seen, palpated or imaged.

As used herein “CTV” or “Clinical Target Volume” is the visible (imaged) or palpable tumor plus any margin of subclinical disease that needs to be eliminated through the treatment planning and delivery process.

The third volume, the planning target volume (PTV), allows for uncertainties in planning or treatment delivery. It is a geometric concept designed to ensure that the radiotherapy dose is actually delivered to the CTV.

Radiotherapy planning must always consider critical normal tissue structures, known as organs at risk “OAR”. In some specific circumstances, it is necessary to add a margin analogous to the PTV margin around an OAR to ensure that the organ cannot receive a higher-than-safe dose; this gives a planning organ at risk volume.

As used herein, a “cold spot” is a decrease of dose to an area significantly under the prescribed dose. While there is no hard fast rule as to what quantifies a cold spot, numbers greater than 10% below prescription should be scrutinized. A “hot spot” is the opposite, an area receiving >10% over prescription.

As used herein, “fractionation” refers to radiation therapy treatments given in daily fractions (segments) over an extended period of time, sometimes up to 6 to 8 weeks.

“High Dose Rate” or “HDR” brachytherapy is the delivery of brachytherapy on an outpatient basis using HDR brachytherapy equipment. The actual treatment delivery last approximately 5-10 minutes in contrast to a hospital stay that might take several days for low-dose rate (LDR) brachytherapy. HDR is almost always done with remote afterloader devices due to the high exposures hospital personnel would receive if they stayed in the room with the patient during administration.

By “inflation” herein what is mean is inflation to the recommended pressure level, thus the volume will vary according to the size of the device, but typically range from 40-70 cc, or about 50-60 or 55 cc for a vaginal balloon, and 80-120 for a rectal balloon.

By “radio-opaque” what is meant is a material that obstructs the passage of radiant energy, such as x-rays, the representative areas appearing light or white on the exposed film. In preferred embodiments, the devices are asymmetrically marked with a radio-opaque material such that placement and orientation can be reproducibly achieved with every treatment.

Polymers used to produce applicators, balloons, jacket materials, caps and the like are commonly filled with substances opaque to x-rays, thereby rendering the devices visible under fluoroscopy or x-ray imaging. These fillers, or radiopacifiers—typically dense metal powders—affect the energy attenuation of photons in an x-ray beam as it passes through matter, reducing the intensity of the photons by absorbing or deflecting them. Because these materials exhibit a higher attenuation coefficient than soft tissue or bone, they appear lighter on a fluoroscope or x-ray film. This visibility provides the contrast needed to accurately position the device in the affected area. Image contrast and sharpness can be varied by the type and amount of radiopacifier used, and can be tailored to the specific application of the device.

Barium sulfate (BaSO₄) was the first radiopaque material to be widely compounded in medical formulations and is the most common filler used with medical-grade polymers because it is very inexpensive at about 2$/lb. Bismuth in another such material, but is more expensive than barium at 20-30$/lb. A fine metal powder with a specific gravity of 19.35, tungsten (W) is more than twice as dense as bismuth and can provide a high attenuation coefficient at a moderate cost of 20$/lb.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim. The term “consisting of” is a closed linking verb, and does not allow the addition of other elements.

The term “consisting essentially of” occupies a middle ground, allowing non-material elements to be added. In this case, these would be elements such as marking indicia, radio-opaque markers, a stopper, packaging, instructions for use, labels, and the like.

The following abbreviations are used herein:

| | |
|---|---|
| ABS | Acrylonitrile butadiene styrene |
| APBI | Accelerated partial breast irradiation |
| CRT | Conformal radiation therapy |
| CT | computer tomography |
| CTV | Clinical Target Volume. |
| DVH | dose-volume histogram |
| EBRT | External beam radiation therapy |
| GTV | Gross tumor volume |
| HDR | High dosage rate |
| IGRT | image guided radio therapy |
| IMRT | intensity-modulated radiation therapy |
| IV | Irradiated volume |
| LDR | Low dosage rate |
| MRI | magnetic resonance imaging |
| OAR | Organ at risk |
| PDR | Pulsed dosage rate |
| PEEK | Polyether ether ketone |
| PET | position emission tomography or polyethylene terephthalate |
| PRV | Planning organ-at-risk volume |
| PTV | Planning target volume |
| PVC | Poly vinyl chloride |
| RVR | Remaining volume at risk |
| TV | Treated volume |
| XRT | radiation therapy |

The foregoing disclosure and description of the invention are illustrative and explanatory thereof, and various changes in the details of the illustrated apparatus and system, and the construction and method of operation may be made without departing from the spirit of the invention.

Each of the following is incorporated by reference herein in its entirety for all purposes:
Wesseling, M., et al., Tackiness of acrylic and cellulosic polymer films used in the coating of solid dosage forms, European Journal of Pharmaceutics and Biopharmaceutics 47(1):73-78 (1999).
US20140221724, US20140221724, U.S. Pat. No. 8,735,828 “Real-time in vivo radiation dosimetry using scintillation detector” by Beddar
US20120281945, US20140367025, U.S. Pat. No. 8,953,912, U.S. Pat. No. 8,885,986 “Small diameter radiation sensor cable” by Isham
US20100288934, US20140018675, US20150216491, U.S. Pat. No. 9,028,390, U.S. Pat. No. 9,351,691, “Apparatus and method for external beam radiation distribution mapping” by Keppel
US20060173233 “Brachytherapy applicator for delivery and assessment of low-level ionizing radiation therapy and methods of use” by Lovoi
WO2003062855 “Method and apparatus for real time dosimetry” by Rosenfeld
US20100318029 Semi-compliant medical balloon
U.S. Pat. No. 4,584,991 Medical device for applying therapeutic radiation
US20150335913 Brachytherapy applicator device for insertion in a body cavity

1. A multichannel brachytherapy applicator and radiation sensor device, comprising:
a) a solid tubular body having a rounded distal end;
b) said solid tubular body having a plurality of hollow radiation lumens, each sized to receive an afterloader cable or a radiation source cable;
c) said solid tubular body having a plurality of hollow sensor lumens, each sized to receive plastic scintillator detector cables; and,
d) each sensor lumen being adjacent one or two radiation lumens to provide one or more lumen pairs or lumen triplets.

2. The device of claim 1, each sensor lumen being within 3 mm of a nearest adjacent radiation lumen.

3. The device of claim 1, each sensor lumen being within 2 mm of a nearest adjacent radiation lumen.

4. The device of claim 1, each sensor lumen being within 1 mm of a nearest adjacent radiation lumen.

5. The device of claim 1, comprised of at least a nose cone component comprising said rounded distal end and a main body component, said nose cone and said main body being operably connected.

6. The device of claim 5, said nose cone comprising an inner nose cone with outer grooves on an exterior surface thereof, and an outer nose cone with inner grooves on an interior surface thereof, said outer grooves aligning with said inner grooves on assembly to form said lumens.

7. The device of claim 1, further comprising a inserter tube component that fits to a proximal end of said main body, said inserter tube having a hollow interior for holding a plurality of cables.

8. The device of claim 5, each component having a male or female end for alignment with a female or male end of an adjacent component on assembly.

9. The device of claim 8, wherein said male or female ends are arranged asymmetrically.

10. The device of claim 5, wherein each component is made of plastic.

11. The device of claim 5, wherein each component is made of polystyrene, polycarbonate/ABS Alloy, PEI, polysulfone, PEEK, acetal or high impact polystyrene nylon.

12. The device of claim 1, wherein said sensor lumens are larger in diameter than said radiation lumens.

13. The device of claim 1, wherein said sensor lumens are larger in diameter than said radiation lumens, except for a larger central radiation lumen.

14. A brachytherapy applicator and sensor device, comprising:
a) a solid tubular body having a rounded distal end;
b) said solid tubular body having one or more hollow radiation lumens sized to receive an afterloader or a radiation source;
c) said solid tubular body having one or more hollow sensor lumens sized to receive an plastic scintillator detector cable; and,
d) each of said sensor lumens having a plastic scintillator detector (PSD) sensor cable therein for measuring radiation dosage.

15. The device of claim 14, wherein said sensor lumens are larger in diameter than said radiation lumens.

16. The device of claim 14, wherein said sensor lumens are larger in diameter than said radiation lumens, except for a centrally located radiation lumen.

17. The device of claim 14, wherein said solid tubular body is made of at least two components: i) a semispherical head portion operably connected to ii) a cylindrical body portion.

18. The device of claim 14, said device further including a semi-compliant balloon surrounding a distal end of said solid tubular body.

19. The device of claim 14, said device further including a non-compliant balloon surrounding a distal end of said solid tubular body.

20. The device of claim 14, wherein said sensor lumens are located near said radiation lumens.

21. The device of claim 14, wherein said sensor lumens are located within 2 mm of said radiation lumens, providing pairs of lumens.

22. The device of claim 14, wherein said sensor lumens are located within 2 mm of a pair of radiation lumens such that said pair of radiation lumens bracket each sensor lumen, providing a triplet of lumens.

23. The device of claim 14, each of said PSD sensor cables having a radio-opaque marker thereon.

24. The device of claim 14, each of said PSD sensor cables having a distal tip, each distal tip having a radio-opaque marker thereon.

25. The device of claim 14, said PSD sensor cables being removably inserted from said sensor lumens.

26. The device of claim 14, said PSD sensor cables being adhered into said sensor lumens.

27. The device of claim 14, each of said PSD sensor cables comprising an opaque jacket enclosing a plastic scintillating fiber at a distal tip directly abutting a fiber optic cable, said fiber optic cable longer than said solid tubular body and terminating in an adaptor for reversible connection to a separate photodetector measuring device.

28. The device of claim 14, each of said PSD sensor cables having a diameter of 2 mm or less.

29. The device of claim 14, said sensor lumens having a smooth low friction surface

30. The device of claim 14, said sensor lumens having coefficient of friction of <0.3.

31. The device of claim 14, said sensor lumens and said PSD sensor cables having a coefficient of friction of <0.2.

32. A brachytherapy treatment method comprising:
a) inserting the device of any claim herein into a body cavity having a tumor;
b) inserting PSD sensor cables into each of said sensor lumens (unless already present);
c) connecting each of said PSD sensor cables to a photodetector measuring device;
d) inserting an afterloader containing a radiation source into one or more of said radiation lumens;
e) treating said tumor and measuring dosage during said treatment;
f) optionally measuring a dose of said radiation source;
g) retracting said afterloader;
h) disconnecting said PSD sensor cables from said photodetector measuring device; and,
i) removing said device from said cavity.

33. A brachytherapy treatment method comprising:
a) inserting the device of claim 23 into a cavity having a tumor;
b) imaging said radio-opaque markers and repositioning said device or said sensor as needed to target a first treatment site on or in said tumor;
c) connecting each of said PSD sensor cables to a photodetector measuring device;
d) inserting an afterloader containing a radiation source into one or more of said radiation lumens;
e) treating said tumor and measuring dosage during said treatment;
f) optionally measuring a dose of said radiation source;
g) optionally repeating steps b-f for a second or more treatment sites;
h) retracting said afterloader;
i) disconnecting said PSD from said photodetector measuring device; and,
j) removing said device from said cavity.

34. The method of claim 33, wherein a depth of said sensor in said sensor lumen is adjusted to a desired level near a depth of said radiation source.

35. A brachytherapy applicator and radiation sensor device, comprising:
a) a solid tubular body having a rounded distal end;
b) said solid tubular body having a central hollow radiation lumen sized to receive an afterloader or a radiation source within 2 mm of a central hollow sensor lumen sized to receive a plastic scintillator detector cable; and,
c) said solid tubular body having one or more peripheral radiation lumens or radiation grooves sized to receive an afterloader or a radiation source, each within 2 mm of a peripheral sensor lumen or sensor groove sized to receive an PSD sensor cable.

36. A brachytherapy applicator and radiation sensor device, comprising:
a) a solid tubular body having a rounded distal end and a proximal end;
b) said solid tubular body having a plurality of pairs or triplets of hollow lumens therein, each lumen opening to an exterior at said proximal end and reaching or nearly reaching said distal end;
c) each pair or triplet of lumens comprising a first radiation lumen sized to receive an afterloader or a radiation source and second or third sensor lumens sized to receive plastic scintillator detector cables, said first radiation lumen less than 3 mm from said second sensor lumen.

37. The device of claim 36, made of plastic.

38. The device of claim 36, made of plastic by injection molding a semispherical distal end and a tubular proximal end, said distal end and said proximal end operably coupled together.

39. The device of claim 36, each sensor lumen comprising a plastic scintillator detector sensor cable for measuring real-time radiation.
